(19) United States
(12) Patent Application Publication
(10) Pub. No.: US 2003/0031725 A1
(43) Pub. Date: Feb. 13, 2003

(54) Composition to restore balance to human physiology and promote healing of skin lesions using mineral salts and catalyst-altered water in an aqueous solution

(76) Inventor: Ray Carl Massner, Prescott, AZ (US)

Correspondence Address: Ray C. Massner, D.C., 2063 Thumb Butte Rd., Prescott, AZ, 86305, US

(21) Appl. No.: 09/966,631

(22) Filed: Oct. 1, 2001

Related U.S. Application Data
(60) Provisional application No. 60/305,775, filed on Jul. 17, 2001.

(51) Int. Cl. A61K 33/00; A61K 33/32; A61K 33/36

(52) U.S. Cl. 424/600; 424/641; 424/717; 424/667; 424/724

(57) ABSTRACT

A safe and effective composition that can be taken orally to restore proper balance in pH and trace mineral levels to an individual who exhibits a need for such adjustment. In one embodiment it may be used topically to promote healing of skin lesions and sores. It contains mineral salts and catalyst-altered water in an aqueous solution and may also contain ethanol.

### CROSS REFERENCE TO RELATED APPLICATIONS

This nonprovisional application claims the benefit of U.S. Provisional Application No. 60/305,775, filed Jul. 17, 2001.

### BACKGROUND OF THE INVENTION

### STATEMENT REGARDING FEDERALLY SPONSORED RESEARCH OR DEVELOPMENT

Not applicable.

### REFERENCE TO A MICROFICHE APPENDIX

Not applicable.

The present invention relates to a composition that is used to restore the balance of the buffering physiology of the body, to correct mineral imbalances, and promote healing of external lesions, sores, and abrasions.

According to research, human physiology operates optimally when the pH values of urine and saliva measure about 6.2-6.4. (See Beddoe, A. F., DDS. “Biologic ionization as applied to human nutrition, principles and techniques” pp.55-91. Oroville, Wash.: S & J Unlimited. 1994.) The normal pH value of blood is 7.35-7.4. (See Guyton, A. C., M.D. “Textbook of medical physiology” p. 111. Philadelphia, Pa.: W. B. Saunders Company. 1961.)

The pH levels of the body relate to the speed of electron spin around the nucleus of the atom. An acidic substance has a counterclockwise spin of rotation and is cationic in nature; it exhibits less resistance so its electrons have a greater rate of spin. Electrons increase their spin tenfold for every degree of acidity. (See Beddoe, A. F., DDS. “Biologic ionization as applied to human nutrition, principles and techniques” pp. 64-5. Oroville, Wash.: S & J Unlimited. 1994.) Therefore when a substance is highly acidic it gives up its electrons easily and tends to disintegrate or oxidize rapidly. In human physiology this increases the degenerative process. (See Beddoe, A. F., DDS. “Biologic ionization as applied to human nutrition, principles and techniques” p. 62. Oroville, Wash.: S & J Unlimited. 1994.) An alkaline substance has a clockwise spin of rotation and is anionic in nature. When a substance is highly alkaline it exhibits a greater resistance, hence there is a slowing of the electrons around the nucleus. This makes it difficult for electrons to be given up or electrons to take their place, reducing chemical activity.

An imbalance in the acid/alkaline relationship, as identified by pH measurement, is thus undesirable and harmful to the health of the individual. (See Guyton, A. C., M.D. “Textbook of medical physiology” p. 111. Philadelphia, Pa.: W. B. Saunders Company. 1961; Beddoe, A. F., DDS. “Biologic ionization in human nutrition, principles and techniques” p. 57. Oroville, Wash.: S & J Unlimited. 1994.) When an individual's urine/saliva pH is acid (cationic), adding calcium and magnesium to the diet will normally reduce the acidity, bringing it closer to the ideal level of 6.4. If an individual's urine/saliva pH is alkaline (anionic), an acid form of calcium and vitamin C can be added to the diet to create acid and make the urine/saliva just slightly acid, pH 6.4. Amphoteric compounds, which when added to a strong acid weaken that acid and added to an alkaline system make it less alkaline, can also be used to make the adjustment.

However, there are many individuals that do not respond to this treatment protocol. The pH of their urine/saliva is about 5.2-5.4. This will improve slightly with increased calcium consumption and the addition of calcium hydroxide, which is strongly alkaline, to the diet, but the pH levels seldom change materially until the physiology of the individual can be changed. This would include the digestive tract, the lymphatic system, the organs, and the biofeedback mechanism. The need in the past has been for a composition that will change the balance between acid and alkaline levels and the balance of trace minerals of the individual slowly but when necessary can be administered in increased dosages without damaging the organism. Also, individuals with such imbalances in pH or trace mineral levels may experience difficulty in healing external lesions, sores, and abrasions. Another need, therefore, has been for a composition that would not only correct the pH and trace mineral imbalances but also would facilitate healing of such external lesions. These needs led to the development of the present composition in all of its embodiments.

### BRIEF SUMMARY OF THE INVENTION

The object of this invention is to restore the balance of the buffering physiology of the body, to correct mineral imbalances, and promote healing of external lesions, sores, and abrasions. The present invention satisfies the need for a composition that will modify the pH of the internal environment slowly but when necessary can be administered in increased dosages without damaging the organism. It can be used to restore a proper balance between acidic and alkaline levels in the body, correct mineral imbalances, and promote external healing.

### BRIEF DESCRIPTION OF THE SEVERAL VIEWS OF THE DRAWING

Not applicable.

### DETAILED DESCRIPTION OF THE INVENTION

In developing this composition the following criteria were used:

1. The composition would contain natural substances that would change the pH of the body without destroying the bacterial flora or causing an increase in oxidation or forming toxic compounds that would damage the immune system.

2. The composition would improve the mechanism of digestion, increasing the glandular secretions of the digestive tract in their appropriate sequence. This would allow for increased absorption of vitamins and minerals. (See Kleiner, I. S., Orten, J. M. “Biochemistry” p. 303. St. Louis, Mo.: The C. V. Mosby Company. 1962.)

3. The composition would promote healing of the intestinal walls and produce an environment that would allow for destruction of pathogenic bacteria, as well as healing external skin lesions such as dermatitis psoriasis, decubitus, and non-healing ulcers caused by staphylococcus and streptococcus infections.

4. The composition would utilize water with a decreased surface tension so that it could be easily transported across the cell membranes and the intestinal flora of the body.

5. The composition would improve the function of the immune system, the organ system, and physiologic function of the body without interfering with other medications, either prescription or over-the-counter drugs.

6. The composition would be in an aqueous solution in an ionic state so the substances making up the embodiments of the composition would remain unaltered chemically and would not decompose within the container. In solution the components would remain ionized and available for consumption in the human body. The dosage would thus be easily regulated to the weight of the individual and for the acuteness and/or duration of the problem by increasing or decreasing the number of drops of solution administered.

7. The composition would be imprintable; i.e., when taken with a homeopathic substance it would not antidote that substance. Rather, it would improve its effectiveness and could act as a carrier of the homeopathic imprint.

The components used in the composition fulfill all of the parameters set forth. Potassium bicarbonate, cesium chloride, and ammonium bicarbonate are all clockwise rotation, making them doubling alkalizers or anionic. Acting amphoterically, in a cationic environment they decrease the acidity and slow the electron spin, whereas in an alkaline environment they weaken the alkalinity by forming more acidic salts. All three compounds ionize easily in water without breaking down chemically. They are all substances occurring naturally within the body and are capable of increasing the glandular secretions and forming ionic solutions that will not destroy intestinal flora. Ammonia neutralizes toxins and destroys bacteria. Ammonium bicarbonate stimulates the production of bile and pancreatic enzymes, which increases the alkalinity of the intestinal tract and colon. This in turn improves the environment of the intestinal flora and promotes healthy bacterial growth. Such bacteria function, along with digestive enzymes, to break down food to provide minerals and release vitamins, especially B-vitamins. Cesium destroys pathogenic bacteria and promotes healing of the intestinal walls. It is also a very strong alkalizer (See Sartori, H. E., M.D. “Nutrients and cancer: an introduction to cesium therapy” pp.7-10; Brewer, A. K., Ph.D. “The high pH therapy for cancer-Tests on mice and humans” pp. 1-5. Pharmacology Biochemistry Behavior; Vol. 21, Suppl. 1. 1984.); its molecular size makes it possible for it to shift the sodium and potassium ratio, improving cellular absorption and neurological transmission. Potassium bicarbonate is also a strong alkalizer. It provides potassium to the body, balancing the sodium-potassium ratio and therefore improving adrenal function.

Other components of the composition include calcium hydroxide, zinc chloride, tincture of silica, cold-processed sea salt, catalyst-altered water, and a small amount of ethanol. Calcium hydroxide is the most readily available form of calcium. It is also a strong alkalizer. Calcium preserves the protoplasm when a cell dies, making it available for new cell growth. It decreases the need for the body to pull calcium from the bone to satisfy its requirements; this helps prevent osteoporosis. Zinc chloride not only provides zinc, which is essential for our immune system, but also changes bacteria, viruses, and fungi from a negative chemotaxis to a positive chemotaxis so the white blood cells can attack and destroy pathogenic bacteria. Tincture of silica acts as a transmuting agent and helps release calcium for use in the body. The cold-processed sea salt carries all the trace minerals in a relationship that is similar to that of human blood. Catalyst-altered water contains calcium chloride, which is a wetting agent and makes water more readily absorbed. The inclusion of small amounts of ethanol not only preserves the products but also improves the imprintability of homeopathic remedies.

The composition consists of mineral salts and catalyst-altered water in an aqueous solution. The mineral salts used in the various embodiments include cesium chloride, ammonium bicarbonate, potassium bicarbonate, iodine, calcium hydroxide, zinc chloride, tincture of silica, and cold-processed sea salt. Ethanol may be present in the composition.

One embodiment of the composition was developed to meet the need to reduce acidity in individuals who display highly acid pH levels of urine and/or saliva. It consists of mineral salts, catalyst-altered water, and ethanol in an aqueous solution. The mineral salt components of this embodiment are potassium bicarbonate, cesium chloride, and ammonium bicarbonate; these are anionic substances. Each mineral salt component is present in an amount of about 3% to about 5% by weight, more preferably 4% to about 4.5% of the composition. The composition has been enhanced by the use of catalyst-altered water in an amount of about 0.1% to about 1% by weight, preferably 0.5% to about 1% by weight of the composition. Catalyst-altered water improves absorption across the cellular membrane. Ethanol is present in an amount of about 3% to about 5% by weight, preferably from 4% to 4.5% by weight of the composition. This embodiment stimulates pancreatic and bile functions, thus aiding the alkalization of the intestinal tract. In such an alkaline environment digestive enzymes also improve their functions. When the composition is taken orally in a suggested dosage, the absorption of these anionic or alkalized substances then aids in reducing the acidity of the blood by forming buffering and amphoteric compounds which gradually shift the pH levels of the individual to a more acceptable range.

Another embodiment of the composition was developed with the object of recreating in human blood the same balance of minerals that exists in sea water, since human blood has been noted to be similar in composition to that of sea water with its mineral and trace mineral content. This embodiment contains mineral salts, iodine, cold-processed sea salt, catalyst-altered water, and ethanol in an aqueous solution. Cesium chloride and ammonium bicarbonate each are present in an amount from about 1% to about 5% by weight, preferably 1% to about 2.5% by weight of the composition. Liquid iodine is also present in an amount from about 0.01% to about 0.2% by weight, preferably 0.03% to about 0.1% by weight of the composition. Cold-processed sea salt is also present in an amount from about 5% to 9% by weight, preferably 6.5% to about 8% by weight of the composition. It provides a harmonic template for the intestinal flora, which break down food and manufacture the vitamins and minerals which are needed for the body. This composition has also been enhanced by the use of catalyst-altered water in an amount from about 0.1% to about 1% by weight, preferably from 0.5% to about 1% by weight of the composition; again this improves absorption across the cellular membrane. Ethanol is present in an amount from about 3% to about 5% by weight, preferably from 4% to about 4.5% by weight of composition. When this embodiment is taken orally in a suggested dosage, the balance of the minerals in the blood of an individual may be restored to more acceptable levels.

Another embodiment of the composition was developed to provide an ionic form of calcium and zinc which can be readily absorbed across the cell membranes of the body without the necessity of combination with chelated or other transmitting substances. A balanced calcium level is necessary to maintain consistent energy in an individual, as it has the effect of preserving protoplasm so that it may be reconstituted for new tissue cell formation. It contains calcium hydroxide, zinc chloride, tincture of silica, catalyst-altered water, and ethanol in an aqueous solution. Calcium hydroxide is present in an amount from about 0.1% to about 2% by weight, preferably 0.3% to about 1% of the weight of the composition. A saturated solution of zinc chloride is present in an amount from about 0.01% to about 0.1% by weight, preferably from 0.01% to about 0.05% by weight of the composition. Tincture of silica is present in an amount from about 0.01% to about 1% by weight, preferably from 0.02% to about 0.05% by weight of the composition. The use of tincture of silica helps release calcium, making it more readily available for use in the body. Catalyst-altered water is present in an amount from about 0.1% to about 2% by weight, preferably from 0.5% to about 1% by weight of the composition. The use of catalyst-altered water in the embodiment aids in absorption of the minerals. Ethanol is present in an amount from about 3% to about 5% by weight, preferably from 4% to about 4.5% by weight of composition. When this embodiment is taken orally in a suggested dosage optimum levels of calcium and zinc may be achieved and maintained.

Another embodiment of the composition was developed to provide a means to promote healing of open lesions and abrasions of the skin. Zinc has long been used as a topical healing agent, but its effectiveness in this embodiment of the composition has been enhanced by its combination with cesium, which can easily cross cellular membranes and strengthen the natural defenses of the cell; ammonium bicarbonate, which helps fight infection; and potassium bicarbonate, which reduces acidity of wounds. A saturated solution of zinc chloride is present in the composition in an amount from about 0.01% to about 0.1% by weight, preferably from 0.01% to about 0.05% by weight of the composition. Potassium bicarbonate, cesium chloride, and ammonium bicarbonate are present in the composition, each in an amount from about 3% to about 6% by weight, preferably from 4% to about 5.5% by weight of the composition. Catalyst-altered water is also present in the composition from about 0.1% to about 2% by weight, preferably from 0.5% to about 1% by weight of the composition. The catalyst-altered water enhances the penetrating ability of this embodiment; thus, applied topically, healing of open lesions, wounds, and abrasions of the skin may be accelerated.

The invention is further illustrated by the following non-limiting examples:

### EXAMPLE 1

A composition of the invention is prepared as follows:

| Material | Weight by percentage |
|---|---|
| Potassium bicarbonate | 4.06% |
| Cesium chloride | 4.06% |
| Ammonium bicarbonate | 4.06% |
| Catalyst-altered water | .68% |
| Ethanol | 4.35% |
| Distilled water | 82.79% |

Dispense half of the distilled water into a container large enough for the purpose. Weigh out each mineral salt; place in a mortar and break up any clumps with a pestle. Place the mineral salts in the container. Add the ethanol and catalyst-altered water to the container; place the lid on the container and shake sufficiently to allow mixture of ingredients. Add the remaining distilled water to the container, cover, and succuse the whole container 100 times. Let the composition sit, covered, for 24-48 hours to allow all ingredients to ameliorate. The composition is now ready to be dispensed.

### EXAMPLE 2

A composition of the invention is prepared as follows:

| Material | Weight by percentage |
|---|---|
| Cold-processed sea salt | 7.44% |
| Cesium chloride | 1.86% |
| Ammonium bicarbonate | 1.86% |
| Iodine (liquid) | .06% |
| Catalyst-altered water | .66% |
| Ethanol | 4.25% |
| Distilled water | 83.87% |

Dispense half the distilled water into a container large enough for the purpose. Weigh out each mineral salt and the sea salt; place in a mortar and break up any clumps with a pestle. Place the salts in the container. Add the ethanol, liquid iodine, and catalyst-altered water to the mixture; place the lid on the container and shake sufficiently to allow mixture of the ingredients. Add the remaining distilled water to the container, cover, and succuse the whole container 100 times. Let the composition sit, covered, for 24-48 hours to allow all ingredients to ameliorate. The composition is now ready to be dispensed.

### EXAMPLE 3

A composition of the invention is prepared as follows:

| Materials | Weight by percentage |
|---|---|
| Calcium hydroxide | .61% |
| Tincture of silica | .37% |
| Zinc chloride | .02% |
| (in saturated solution) | |
| Catalyst-altered water | .74% |
| Ethanol | 4.74% |
| Distilled water | 93.52% |

Dispense half the distilled water into a container large enough for the purpose. Weigh out the calcium hydroxide. Place into a mortar and break up any clumps with a pestle. Place the calcium hydroxide in the container. Add the zinc chloride solution, the tincture of silica, ethanol, and catalyst-altered water to the mixture; place the lid on the container and shake sufficiently to allow mixture of the ingredients. Add the remaining distilled water to the container, cover, and succuse the whole container 100 times. Let the composition sit, covered, for 24-48 hours to allow all ingredients to ameliorate. The composition is now ready to be dispensed.

### EXAMPLE 4

A composition of the invention is prepared as follows:

| Materials | Weight by percentage |
|---|---|
| Potassium bicarbonate | 4.31% |
| Cesium chloride | 4.31% |
| Ammonium bicarbonate | 4.31% |
| Zinc chloride | .02% |
| (in saturated solution) | |
| Catalyst-altered water | .64% |
| Distilled water | 86.41% |

Dispense half of the distilled water into a container large enough for the purpose. Weigh out each mineral salt. Place into a mortar and break up any clumps with a pestle. Place the mineral salts in the container. Add the zinc chloride and the catalyst-altered water to the mixture; place the lid on the container and shake sufficiently to allow mixture of the ingredients. Add the remaining distilled water to the container, cover, and succuse the whole container 100 times. Let the composition sit, covered, for 24-48 hours to allow all ingredients to ameliorate. The composition is now ready to be dispensed.

Although the present invention has been described in detail in the foregoing specification with respect to various embodiments, such specification is intended to be illustrative only and not limiting. It will be apparent to those of ordinary skill in the art that many alternatives, equivalents, and variations will fall within the spirit and the scope of the present invention without departing from its principles.

I claim:

1. A composition to restore the balance of the buffering physiology of the human body, to correct mineral imbalances, and promote healing of external lesions, comprising an aqueous base containing:
(a) catalyst-altered water in an amount not greater than 2% by weight of the composition; and
(b) at least two components selected from the group consisting of potassium bicarbonate, cesium chloride, ammonium bicarbonate, iodine, calcium hydroxide, tincture of silica, and zinc chloride.

2. The composition of claim 1 in which (b) is potassium bicarbonate in an amount not greater than 5% by weight of the composition, cesium chloride in an amount not greater than 5% by weight of the composition, and ammonium bicarbonate in an amount not greater than 5% by weight of the composition.

3. The composition of claim 2 which also comprises ethanol in an amount not greater than 5% by weight of the composition.

4. The composition of claim 1 in which (b) is iodine in a liquid form in an amount not greater than 0.2% by weight of the composition, cesium chloride in an amount not greater than 5% by weight of the composition, and ammonium bicarbonate in an amount not greater than 5% by weight of the composition.

5. The composition of claim 4 which also comprises a cold-pressed sea salt in an amount of no more than 9% by weight of the composition.

6. The composition of claim 4 which also comprises ethanol in an amount no greater than 5% by weight of the composition.

7. The composition of claim 1 in which (b) is calcium hydroxide in an amount not greater than 2% by weight of the composition, zinc chloride in an amount not greater than 0.1% by weight of the composition, tincture of silica in an amount no greater than 1% by weight of the composition.

8. The composition of claim 7 which also comprises ethanol in an amount no greater than 5% by weight of the composition.

9. The composition of claim 1 in which (b) is potassium bicarbonate in an amount not greater than 6% by weight of the composition, cesium chloride in an amount not greater than 6% by weight of the composition, ammonium bicarbonate in an amount not greater than 6% by weight of the composition, and zinc chloride in an amount not greater than 0.1% by weight of the composition.
